(19) United States
(12) Patent Application Publication
(10) Pub. No.: US 2022/0348981 A1
(43) Pub. Date: Nov. 3, 2022

(54) ASSAY OF ARYLSULFATASE A ENZYMATIC ACTIVITY IN DRIED BLOOD SPOTS BY MASS SPECTROMETRY AND FLUOROMETRY

(71) Applicant: University of Washington, Seattle, WA (US)

(72) Inventors: Michael H. Gelb, Seattle, WA (US); Xinying Hong, Seattle, WA (US)

(73) Assignee: University of Washington, Seattle, WA (US)

(21) Appl. No.: 17/762,324

(22) PCT Filed: Sep. 18, 2020
(86) PCT No.: PCT/US2020/051455

§ 371 Date: Mar. 21, 2022

Related U.S. Application Data
(60) Provisional application No. 62/904,492, filed on Sep. 23, 2019.

(51) Int. Cl. C12Q 1/44

(52) U.S. Cl. CPC C12Q 1/44; G01N 2800/10

(57) ABSTRACT

Methods, reagents, and kits for assaying for arylsulfatase A activity for diagnosing conditions associated with arylsulfatase A deficiency, such as multiple sulfatase deficiency (MSD) and metachromatic leukodystrophy (MILD).

### CROSS-REFERENCE TO RELATED APPLICATION

This application claims the benefit of U.S. Application No. 62/904,492, filed Sep. 23, 2019, expressly incorporated herein by reference in its entirety.

### STATEMENT OF GOVERNMENT LICENSE RIGHTS

This invention was made with government support under Grant No. R01 DK067859 awarded by the National Institutes of Health. The government has certain rights in the invention.

### BACKGROUND OF THE INVENTION

Metachromatic leukodystrophy (MLD) is caused by deficiency of the lysosomal enzyme arylsulfatase A (ARSA) which removes sulfate from 3-sulfo-galactosylceramides (sulfatides). With the development of potential treatments, including gene therapy and hematopoietic stem cell transplantation, and the demonstration that clinical outcomes are optimized if treatments are initiated prior to full onset of symptoms, newborn screening (NBS) for MLD may be warranted in the near future. Elevated sulfatides in urine and blood are typically seen in MLD patients but not in those carrying pseudodeficiency variants. The diagnosis of MLD also relies on a nominally low ARSA enzymatic activity in leukocyte or fibroblast lysate. However, there have been multiple reports detailing the pitfalls of the current ARSA assays using generic sulfatase substrates.

Recently, after screening approximately 50,000 random newborns in de-identified research NBS study for MLD by measuring sulfatides abundance in dried blood spots (DBS), it became apparent that a second-tier test was required to reduce the number of false positives.

Despite the development ARSA assays for the purpose of diagnosing MLD, needs exist for a sensitive and specific enzymatic assay for ARSA that would be an improvement to the current diagnostic test and for an assay to measure ARSA enzymatic activity in DBS, so that the test can be performed onsite as part of the screening process without the need to contact families to obtain a different sample specimen, such as whole blood or urine. The present invention seeks to fulfill these needs and provides further related advantages.

### SUMMARY OF THE INVENTION

The present invention provides methods, reagents, and kits for assaying for arylsulfatase A activity for diagnosing conditions associated with arylsulfatase A deficiency, such as multiple sulfatase deficiency (MSD) and metachromatic leukodystrophy (MLD).

In one aspect, the invention provides a method for assaying for arylsulfatase A. In certain embodiments, the method comprises:
(a) contacting a sample with a first solution to provide a first solution comprising arylsulfatase A;
(b) isolating arylsulfatase A from the first solution by size exclusion chromatography;
(c) contacting the isolated arylsulfatase A with an arylsulfatase A substrate and incubating the substrate with arylsulfatase A for a time sufficient to provide a solution comprising an arylsulfatase A enzyme product; and
(d) determining the quantity of the arylsulfatase A enzyme product.

In another aspect, the invention provides a method for assaying for arylsulfatase A. In certain embodiments, the method comprises:
(a) contacting a sample with a solution to provide a solution comprising arylsulfatase A;
(b) contacting the solution comprising arylsulfatase A with an arylsulfatase A substrate (e.g., an isotopically-labeled arylsulfatase A substrate or a non-isotopically-labeled arylsulfatase A substrate) and incubating the substrate with arylsulfatase A for a time sufficient to provide a solution comprising an arylsulfatase A enzyme product; and
(c) determining the quantity of the arylsulfatase A enzyme product.

In a further aspect, the invention provides a method for screening a newborn for a condition associated with arylsulfatase A deficiency, such as metachromatic leukodystrophy (MLD) or multiple sulfatase deficiency (MSD). In certain embodiments, the method comprises:
(a) quantifying the amount of sulfatide in a dried blood spot from a newborn using liquid-chromatography tandem mass spectrometry to determine whether the amount of sulfatide is abnormal; and
(b) determining the arylsulfatase A activity in the dried blood spot from the newborn having an abnormal amount of sulfatide, comprising contacting a solution comprising arylsulfatase A from the dried blood spot with an arylsulfatase A substrate and incubating the substrate with arylsulfatase A for a time sufficient to provide a solution comprising an arylsulfatase A enzyme product; and determining the quantity of the arylsulfatase A enzyme product.

The above methods are useful for determining arylsulfatase A activity in biological samples. Samples useful in the above methods includes samples containing arylsulfatase A and that can be analyzed by the methods described herein to determine arylsulfatase A activity. Representative samples include dried blood spots, whole blood, plasma, blood leukocytes, cerebrospinal fluid, and tissues. In the above methods, determining the quantity of the arylsulfatase A enzyme product includes mass spectrometric analysis.

In another aspect of the invention, reagents (substrates and internal standards) for assaying arylsulfatase A are provided.

In a further aspect of the invention, kits for assaying arylsulfatase A are provided. The kits includes one or more reagents (e.g., substrate and internal standards) of the invention.

The invention also provides fluorescence-based arylsulfatase A assays. In certain embodiments, the method for assaying for arylsulfatase A comprises:
(a) contacting a sample with a first solution to provide a first solution comprising arylsulfatase A;
(b) isolating arylsulfatase A from the first solution;
(c) contacting the isolated arylsulfatase A with an arylsulfatase A substrate and incubating the substrate with arylsulfatase A for a time sufficient to provide a solution comprising a fluorescent arylsulfatase A enzyme product; and
(d) determining the quantity of the fluorescent arylsulfatase A enzyme product.

The methods described herein are useful for quantifying the arylsulfatase A enzyme product and can be used to determine whether the sample is from a candidate for treatment for a condition associated with arylsulfatase A deficiency, such as multiple sulfatase deficiency (MSD) and metachromatic leukodystrophy (MLD). Accordingly, the above methods are useful for diagnosing multiple sulfatase deficiency (MSD) or for diagnosing metachromatic leukodystrophy (MLD).

### DESCRIPTION OF THE DRAWINGS

The foregoing aspects and many of the attendant advantages of this invention will become more readily appreciated as the same become better understood by reference to the following detailed description, when taken in conjunction with the accompanying drawings.

FIGS. 1A-1C illustrate LC-MS/MS chromatographs of the ARSA substrate (FIG. 1A); ARSA enzymatic product (FIG. 1B); and ARSA internal standard channel (FIG. 1C). The asterisk in the ARSA enzymatic product channel was from substrate breakdown in the heated ESI source. The x-axis is time (min) and the y-axis is ion counts in the MRM channel after being normalized to the maximum signal (100%).

FIG. 2 shows the amount of ARSA enzymatic product formed as a function of the amount of protein in the leukocyte lysate used. Error bars are standard deviations based on triplicate measurement.

FIG. 3 shows ARSA activity as a function of the fraction of ARSA-containing lymphoblast lysate (GM14603) added to ARSA-deficient lymphoblast lysate (GM23097). A total of 2 μg protein was used per assay. Error bars are standard deviations based on the triplicate measurements. The insert is an expansion of the plot at the lower end.

FIG. 4 compares ARSA activity in DBS from 4 MLD patients (median: 0.007 μM/h, range: 0.005-0.011 μM/h), 1 MSD patient (0.087 μM/h), and 7 healthy adults (0.63 μM/h, range: 0.39-1.30 μM/h), after immuno-precipitation purification. The horizontal bar indicates the median of each group.

FIG. 5 compares ARSA activity after size-exclusion chromatography purification in CDC Quality Control DBS samples, including base pool (0.023±0.003 μM/h), low (0.048±0.007 μM/h), medium (0.21±0.02 μM/h), and high (0.37±0.02 μM/h) controls, each representing 0, 5%, 50% and 100% to high control, respectively. Each point was measured in 20 replicates. Error bars were standard deviations based on the 20 measurements.

FIG. 6A compares ARSA activity after size-exclusion chromatography purification in DBS from 34 MLD patients (median: 0.0015 μM/h, range: 0-0.18 μM/h), 3 MSD patients (median: 0.032 μM/h, range: 0.028-0.076 μM/h), 10 healthy adults (median: 0.80 μM/h, range: 0.45-1.3 μM/h) and 294 random newborns (median: 0.27 μM/h, range: 0.082-0.65 μM/h). Fresh DBS from patients and healthy adults were used. FIG. 6B compares ARSA activity in aged DBS (stored at room temperature for 1 month before processing) from 18 MLD patients (median: 0.003 μM/h, 0-0.043 μM/h), 2 MSD patients (0.021 and 0.035 μM/h), and 205 random newborns (median: 0.21 μM/h, 0.073-0.56 μM/h. The horizontal bar in each figure indicates the median of each group.

FIGS. 7A-7C illustrate the sulfatide analysis in DBS. FIG. 7A shows a UPLC-MS/MS chromatogram of four sulfatide species in DBS from a random newborn. The x-axis is time (min) and the y-axis is the MS/MS intensity. Peak 1 at 1.22 min is C16:1-OH-sulfatide, peak 2 at 1.31 min is C16:0-OH-sulfatide, peak 3 at 1.35 min is C16:0-sulfatide and peak 4 at 1.46 min is C18:0-sulfatide. FIG. 7B shows the C16:0-sulfatide abundance (μM) in DBS from 15 MLD newborns (median: 0.32 μM, range: 0.18-0.47 μM) and 2000 random newborns (median: 0.094 μM, range: 0.020-0.23 μM). The solid line is the median of the MLD group. The dash line is the screening cut-off at 0.17 μM. The crosses indicate MLD newborns without normalized C16:0-sulfatide data available. The circles indicate MLD newborns with normalized C16:0-sulfatide data available and the data are presented in FIG. 7C. FIG. 7C shows the normalized C16:0-sulfatide level in DBS from 6 MLD newborns (median: 1.24, range: 0.68-1.48) and 2000 random newborns (median: 0.34, range: 0.11-0.86). The dash line is the screening cut-off at 0.64 after normalization.

FIGS. 8A and 8B schematically illustrate algorithms for the newborn screening of MLD. FIG. 8A illustrates the C16:0-sulfatide DBS assay is the primary screening test and the ARSA DBS activity assay is the secondary test; FIG. 8B illustrates the ARSA DBS activity assay is the primary screening test and the C16:0-sulfatide DBS assay is the secondary test.

FIG. 9 shows the total sulfatide abundance (μM) in DBS from 15 MLD newborns (median: 0.79 μM, range: 0.50-1.23 μM) and 2000 random newborns (median: 0.25 μM, range: 0.085-0.56 μM). The lines indicate the median of each group.

### DETAILED DESCRIPTION OF THE INVENTION

The present invention provides, in certain embodiments, an ARSA enzymatic activity assay in DBS, which is conducted without an anti-ARSA antiserum. The present invention also provides, in certain embodiments, an in vitro assay to measure ARSA activity in leukocytes lysate that utilizes its natural substrate for specificity. The ARSA leukocyte assay of the invention has an exceptionally high sensitivity and precision such that trace amounts of residual enzymatic activity could be detected with statistical significance. The ARSA assays of the invention are useful for diagnosing patients with multiple sulfatase deficiency (MSD), where defect in the formylglycine generating enzyme prohibits the crucial modification on the active site of all sulfatases.

The assays of the present invention are liquid chromatography-tandem mass spectrometry (LC-MS/MS) assays for measuring arylsulfatase A (ARSA) activity in leukocytes and dried blood spots (DBS) using deuterated natural sulfatide substrate. These assays are highly specific and sensitive. In the assays, patients with metachromatic leukodystrophy (MLD) and multiple sulfatase deficiency (MSD) displayed clear deficit in the enzymatic activity and could be completely distinguished from normal controls. The leukocyte assay of the invention is important for diagnosing MLD and MSD patients and for monitoring the efficacy of therapeutic treatments. The ARSA assay of the invention is the first assay to measure ARSA activity in DBS without the use of an antibody. This ARSA DBS assay can serve as a second-tier test following the sulfatide measurement in DBS for newborn screening of MLD because the ARSA DBS assay leads to the elimination of most of the false positives identified by the sulfatide assay.

In one aspect, the invention provides a method for assaying for arylsulfatase A. In certain embodiments, the method comprises:
(a) contacting a sample with a first solution to provide a first solution comprising arylsulfatase A;
(b) isolating arylsulfatase A from the first solution by size exclusion chromatography;
(c) contacting the isolated arylsulfatase A with an arylsulfatase A substrate and incubating the substrate with arylsulfatase A for a time sufficient to provide a solution comprising an arylsulfatase A enzyme product; and
(d) determining the quantity of the arylsulfatase A enzyme product.

In certain embodiments of the method, the sample is a dried blood spot (e.g., newborn dried blood spot). In other embodiments, the sample is a whole blood sample, a plasma sample, a blood leukocyte sample, a cerebrospinal fluid sample, or a tissue sample.

In another aspect, the invention provides a method for assaying for arylsulfatase A. In certain embodiments, the method comprises:
(a) contacting a sample with a solution to provide a solution comprising arylsulfatase A;
(b) contacting the solution comprising arylsulfatase A with an arylsulfatase A substrate (e.g., an isotopically-labeled arylsulfatase A substrate or a non-isotopically-labeled arylsulfatase A substrate) and incubating the substrate with arylsulfatase A for a time sufficient to provide a solution comprising an arylsulfatase A enzyme product; and
(c) determining the quantity of the arylsulfatase A enzyme product.

In certain embodiments of the method, the sample is a whole blood sample, a plasma sample, a blood leukocyte sample (e.g., leukocyte lysate), a cerebrospinal fluid sample, or a tissue sample.

In a further aspect, the invention provides a method for screening a newborn for a condition associated with arylsulfatase A deficiency, such as metachromatic leukodystrophy (MLD) or multiple sulfatase deficiency (MSD). In certain embodiments, the method comprises:
(a) quantifying the amount of sulfatide in a dried blood spot from a newborn (e.g., using liquid-chromatography tandem mass spectrometry) to determine whether the amount of sulfatide is abnormal; and
(b) determining the arylsulfatase A activity in the dried blood spot from the newborn having an abnormal amount of sulfatide, comprising contacting a solution comprising arylsulfatase A from the dried blood spot with an arylsulfatase A substrate and incubating the substrate with arylsulfatase A for a time sufficient to provide a solution comprising an arylsulfatase A enzyme product; and determining the quantity of the arylsulfatase A enzyme product.

The second-tier of the two-tier assay described above is useful to identify false positives that result from a sulfatide assay conducted on DBS in newborn screening of MLD.

In the above method, the amount of sulfatide in a dried blood spot is determined to be “abnormal” when the amount of sulfatide is greater than or equal to a pre-determined amount (e.g., ≥10 μM, ≥15 μM, ≥17 μM, or 20 μM in blood) (e.g., normalized C16:0-sulfatide level above 0.64, which corresponds to 17 μM in blood). See two-tier arylsulfatase assay and quantifying the amount of sulfatide in a dried blood spot described below.

The dried blood spot used in determining the arylsulfatase activity is from the same newborn for which the amount of sulfatide was quantified and determined to be abnormal. The dried blood spot used for determining the arylsulfatase activity may be from the same dried blood spot used for quantifying sulfatide (each newborn dried blood spot can provide 5-10 samples for analysis) or a second dried blood spot from the same newborn.

In certain embodiments of the method, the sulfatide is C16:0-sulfatide.

In certain embodiments of the method, determining the arylsulfatase A activity comprises the method described above (i.e., size exclusion chromatograph to isolate arylsulfatase A from a dried blood spot, incubating the isolated enzyme with an arylsulfatase A substrate, and quantitating the amount of arylsulfatase A product).

The methods described above are useful for determining arylsulfatase A activity in biological samples. Samples useful in the above methods includes samples containing arylsulfatase A and that can be analyzed by the methods described herein to determine arylsulfatase A activity. Representative samples include dried blood spots, whole blood, plasma, blood leukocytes, cerebrospinal fluid, and tissue samples.

In the methods describe above, determining the quantity of the arylsulfatase A enzyme product includes mass spectrometric analysis. In certain embodiments, determining the quantities of the arylsulfatase A enzyme product includes determining the ratio of each product to its internal standard by mass spectrometric analysis. In certain embodiments, determining the quantity of the arylsulfatase A enzyme product includes tandem mass spectrometric analysis in which the parent ions of the products and their internal standards are generated, isolated, and subjected to collision-induced dissociation to provide product fragment ions and internal standard fragment ions. In certain embodiments, determining the quantities of the arylsulfatase A enzyme product includes comparing the peak intensities of the product fragment ions and internal standard fragment ions to calculate the amount of the product. In certain embodiments, determining the quantity of the arylsulfatase A enzyme product includes conducting the product to a mass spectrometer by liquid chromatography or by flow injection.

In certain of the mass spectrometric methods, the arylsulfatase A substrate is an isotopically-labeled (e.g., deuterium-labeled) substrate. In certain embodiments, the isotopically-labeled substrate is an isotopically-labeled natural arylsulfatase substrate. In certain embodiments, the arylsulfatase A substrate is a deuterated arylsulfatase A substrate. In certain of these embodiments, the arylsulfatase A substrate is d₃-C18:0-sulfatide. In certain of these embodiments, the arylsulfatase A enzyme product is d₃-C18:0-galactosyl-ceramide.

In certain of the mass spectrometric methods, the arylsulfatase A substrate is a non-isotopically-labeled substrate (e.g., a natural arylsulfatase substrate).

In certain of the mass spectrometric methods, the assay further comprises an arylsulfatase A internal standard (e.g., the solution comprising arylsulfatase A substrate further comprises an arylsulfatase A internal standard). In certain of these embodiments, the arylsulfatase A internal standard is an isotopically-labeled internal standard. In certain embodiments, the arylsulfatase A internal standard is a deuterated arylsulfatase A internal standard. In certain of these embodiments, the arylsulfatase A internal standard is d₇-C18:0-galactosyl-ceramide. In certain embodiments of the above methods, an internal standard for arylsulfatase A is added before, after, or simultaneously with contacting the solution comprising arylsulfatase A with the substrate. In certain embodiments of the above methods, the enzyme reaction is quenched prior to determining the quantities of the arylsulfatase A enzyme product.

In certain of the mass spectrometric methods, the arylsulfatase A substrate is d₃-C18:0-sulfatide and the arylsulfatase A enzyme product is d₃-C18:0-galactosyl-ceramide.

In certain embodiments of the above methods, the method further includes using the quantity of the arylsulfatase A enzyme product to determine whether the sample is from a candidate for treatment for a condition associated with arylsulfatase A deficiency. Conditions associated with arylsulfatase A deficiency include multiple sulfatase deficiency (MSD) and metachromatic leukodystrophy (MLD). Accordingly, the above methods are useful for diagnosing multiple sulfatase deficiency (MSD) or for diagnosing metachromatic leukodystrophy (MLD).

In another aspect of the invention, reagents (substrates and internal standards) for assaying arylsulfatase A are provided.

In a further aspect of the invention, kits for assaying arylsulfatase A are provided. In one embodiment, the kit includes one or more reagents (e.g., substrate and internal standard) of the invention.

In one embodiment, the kit comprises an arylsulfatase A substrate. Suitable substrates include isotopically-labeled substrates (e.g., isotopically-labeled natural substrates). In certain embodiments, the arylsulfatase A substrate is a deuterium-labeled substrate. In one embodiment, the arylsulfatase A substrate is d₃-C18:0-sulfatide.

In another embodiment, the kit further comprises an internal standard for arylsulfatase A. Suitable internal standards include isotopically-labeled internal standards. In certain embodiments, the arylsulfatase A internal standard is a deuterium-labeled internal standard. In one embodiment, the arylsulfatase A internal standard is d₇-C18:0-galactosyl-ceramide.

In a further aspect, the invention provides fluorescence-based arylsulfatase A assays. In certain embodiments, this method for assaying for arylsulfatase A comprises:
(a) contacting a sample with a first solution to provide a first solution comprising arylsulfatase A;
(b) isolating arylsulfatase A from the first solution;
(c) contacting the isolated arylsulfatase A with an arylsulfatase A substrate and incubating the substrate with arylsulfatase A for a time sufficient to provide a solution comprising a fluorescent arylsulfatase A enzyme product; and
(d) determining the quantity of the fluorescent arylsulfatase A enzyme product.

In the method, determining the quantities of the fluorescent arylsulfatase A enzyme product comprises fluorescence analysis.

In certain embodiments, isolating arylsulfatase A comprises size-exclusion chromatography.

In certain embodiments, the sample is a dried blood spot. In other embodiments, the sample is a blood leukocyte sample, a whole blood sample, a plasma sample, a cerebrospinal fluid sample, or a tissue sample.

In certain embodiments, the arylsulfatase A substrate is p-nitrocatechol sulfate or 4-methylumbelliferyl sulfate.

In certain embodiments, the method further comprises using the quantity of the fluorescent arylsulfatase A enzyme product to determine whether the sample is from a candidate for treatment for a condition associated with arylsulfatase A deficiency (e.g., multiple sulfatase deficiency (MSD) or metachromatic leukodystrophy (MLD)).

The following is a description of representative ARSA assays for measuring ARSA activity, including in DBS for the first time without an antibody, and a description of representative leukocyte assays for diagnosing MLD and MSD patients and for monitoring the efficacy of therapeutic treatments and.

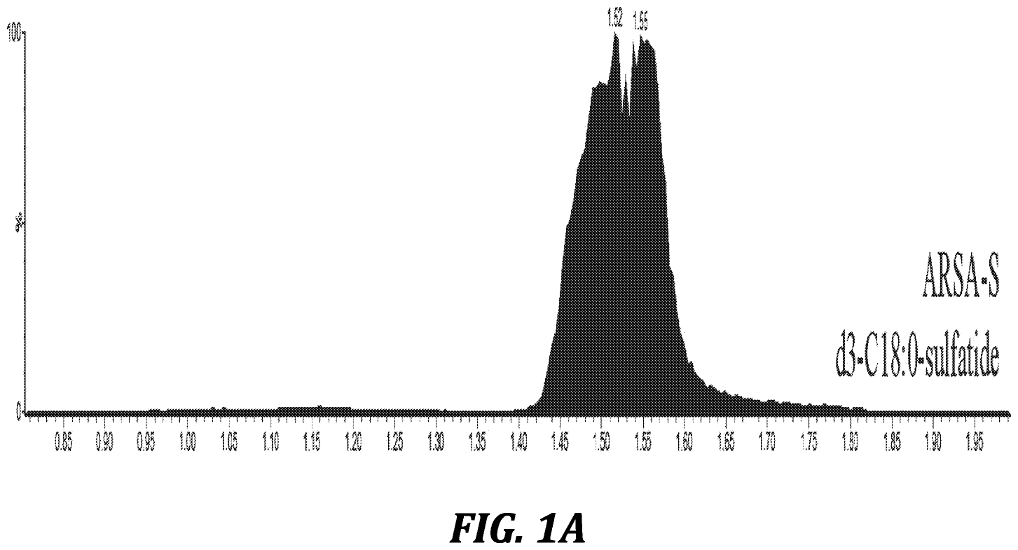

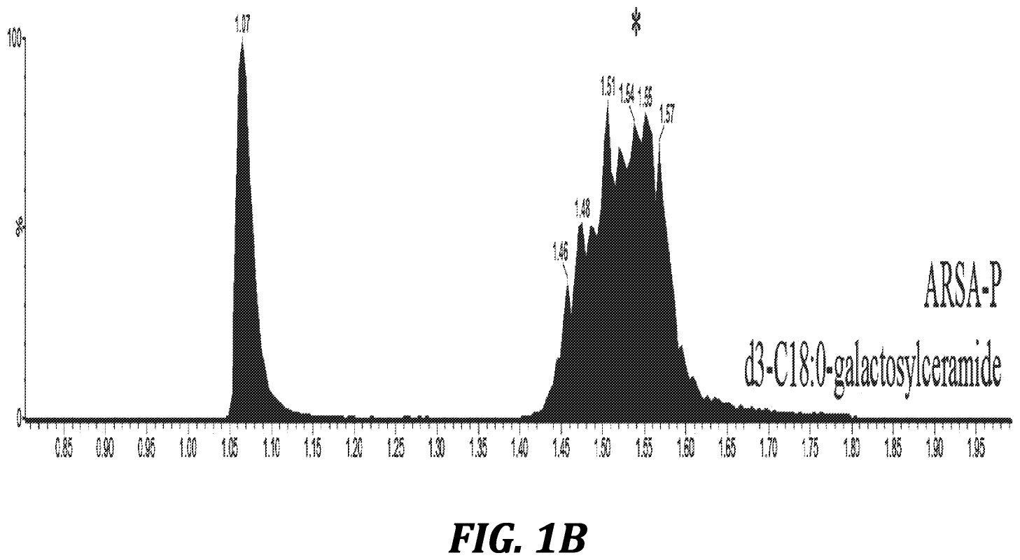

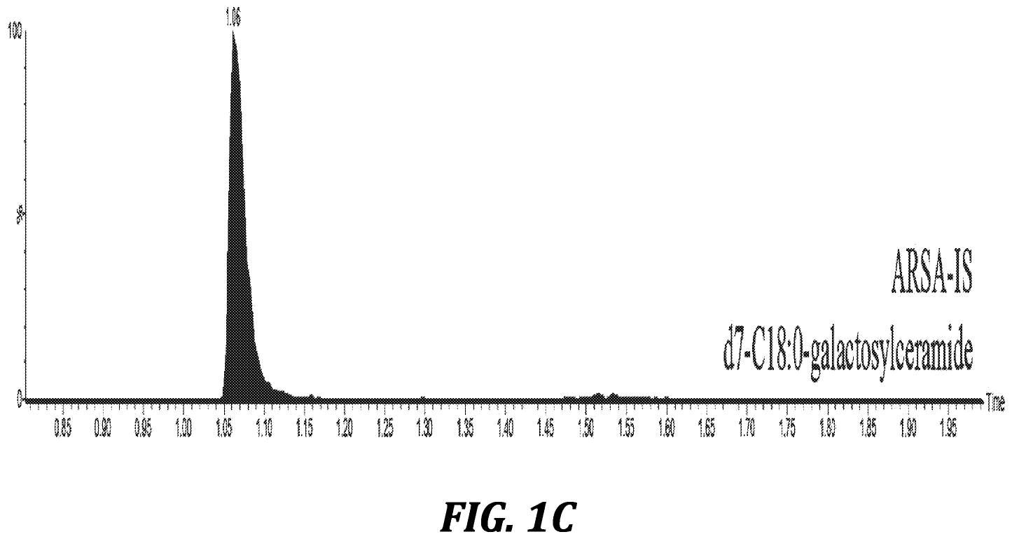

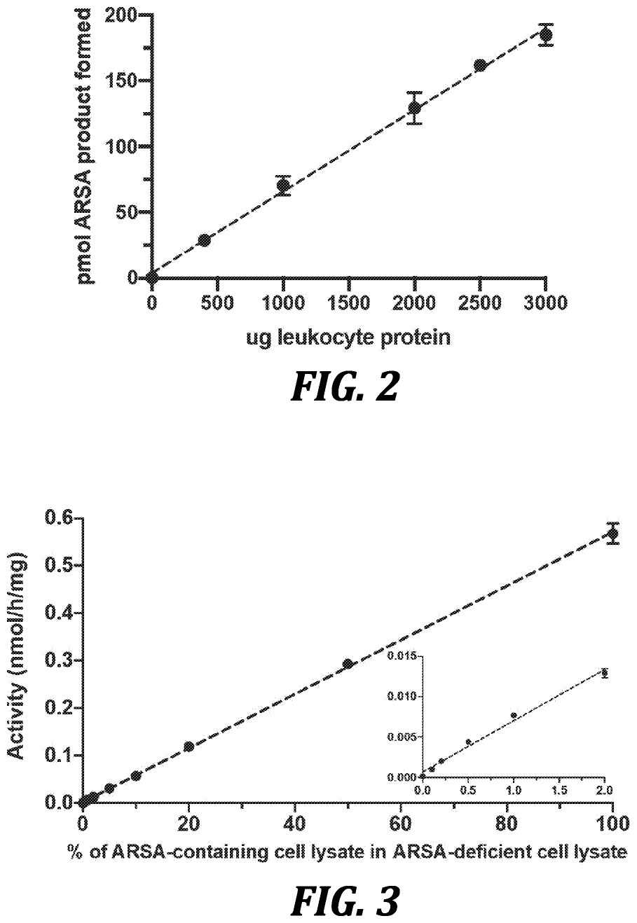

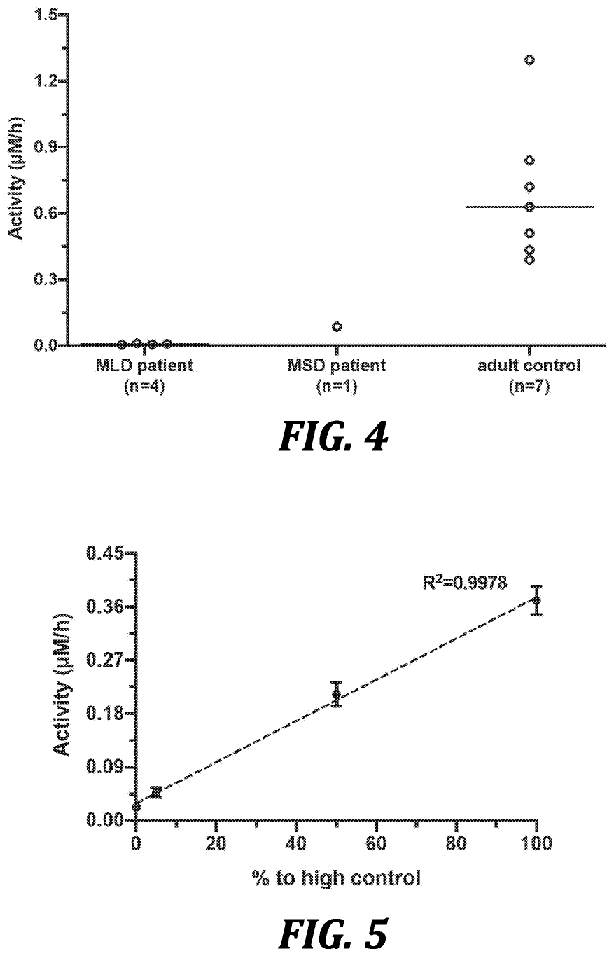

Development of a new ARSA leukocyte assay. Recently, Han et al. reported the detection of ARSA enzymatic activity where a natural substrate, C18:0-sulfatide, was incubated with leukocyte lysate in buffer containing sodium taurodeoxycholate as detergent (Han, M.; Jun, S. H.; Song, S. H.; Park, H. D.; Park, K. U.; Song, J., Ultra-performance liquid chromatography-tandem mass spectrometry measurement of leukocyte arylsulfatase A activity using a natural substrate. Ann Lab Med 2015, 35 (1), 165-8). As described herein, in the present assay, the deuterium labeled sulfatide d₃-C18:0-sulfatide was used as substrate so that the enzymatic product (d₃-C18:0-galactosyl-ceramide) also carried the three deuterium labels, and could be selectively quantified without being interfered by the endogenous galactosyl-ceramide. Using this approach, the assay is able to detect ARSA enzymatic activity in lysates of mixed leukocytes (mainly lymphocytes) by LC-MS/MS. As shown in FIG. 1, the de-sulfated product peak eluted at 1.07 min, and was fully separated from the peak of the sulfated substrate eluting at 1.5 min. If leukocyte lysate was omitted, no discernable product peak was observed above the baseline noise (data not shown). The ARSA activity showed a sigmoidal dependence on the detergent concentration, and a concentration of 2.0 g/L sodium taurodeoxycholate gave maximal activity and was used in all of the following studies. Because detergent was present in the assay buffer, MLD patients that are due to dysfunctional Saposin B protein will not be detected with this assay, but these variants are very rare among MLD patients (less than 5%). In the Han et al. report, MnCl₂ was added to the buffer at a concentration of 33 mM. This was based on earlier findings suggesting that this metal ion lowers the critical micellar concentration of the inhibitory ionic form of the bile salt and allows the formation of mixed micelles of taurodeoxycholate and sulfatide. However, ARSA activity was found to decrease with increasing amount of MnCl₂ in assay buffer, thus MnCl₂ was omitted from the final assay conditions in the present methods. Barium or cerium sulfates are often used to assay sulfatases in complex matrices to sequester inorganic sulfate and phosphate ions, which are potent inhibitors of sulfatases. However, ARSA activity was found to decrease as the concentration of Ce(acetate)₃ was increased from 0 to 20 mM, therefore this salt was omitted from the assay. The ARSA activity was pH dependent with an optimal pH at 4.5. The enzymatic rate displayed hyperbolic kinetics as the substrate concentration varied, giving a K_{M} of 83 μM (FIG. 4). The pH optimum of ARSA and the K_{M} value found agreed with the previous reports. The reaction progress curve shows a falloff from linearity over 20 hours. Even though the progress curve is not linear, the amount of enzymatic product formed after 16 hours of incubation increased linearly with the amount of leukocyte protein used (FIG. 2).

The stability of ARSA in whole blood was studied under optimized assay conditions. K₂EDTA-treated venous blood was kept at room temperature or 4° C. for up to 4 days prior to the preparation of leukocytes. About 14% and 32% of the ARSA activity was lost over the first 24 hours when the whole blood was stored at 4° C. and room temperature, respectively. The ARSA activity continued to decrease with blood storage time under both conditions, with approximately 45% left after 4 days. It is therefore recommended that whole blood be shipped overnight at 4° C. followed by immediate leukocyte preparation and freezing of the cell pellet. ARSA activity decreased only slightly with the number of freeze/thaw cycles of the mixed leukocytes, with about 20% activity lost after 5 cycles.

To study the minimal residual ARSA activity that can be detected by this LC-MS/MS assay, various amount of lysate from ARSA-containing lymphoblasts (Coriell Institute Repository, GM14603) was mixed with lysate from ARSA-deficient lymphoblasts (GM23097) (2 μg protein total). With 100% ARSA-deficient cells lysate, no ARSA activity was observed (FIG. 3). ARSA activity increased proportionally with the percentage of ARSA-containing cells lysate in the mixture. From the inset of FIG. 3, it was clear that as little as 0.1% residual ARSA activity could be detected above baseline with statistical significance.

ARSA activity in leukocyte lysates from MLD and MSD patients. Table 1 shows the ARSA activity in leukocyte lysates from 22 MLD patients and 1 MSD patient. Genotype information and age of onset (categorized by gross motor or cognitive symptoms), if available, are also provided.

TABLE 1
Summary of ARSA activity in leukocyte lysates from 22 MLD patients and 1 MSD patient¹.

| | | | ARSA activity | % to adult | |
|---|---|---|---|---|---|
| | Genotype | age of onset | (nmol/h/mg protein) | control | Note |
| MLD patient 1 | | late infantile | 0.0011 ± 0.0005 | 0.03 | |
| MLD patient 2 | c.370G > A + c*96A > G//c.685- | late infantile | 0.0071 ± 0.0002 | 0.18 | |
| | 1G > A + c.1055A > G | | | | |
| MLD patient 3 | c.465 + 1G > A//c.1108-3C > G | late infantile | 0.0055 ± 0.0007 | 0.14 | |
| MLD patient 4 | c.449C > T//c.474C > A | 12 month | 0.0067 ± 0.0002 | 0.17 | |
| MLD patient 5 | c.178C > T//c.178 C > T | 13 month | 0.0081 ± 0.0007 | 0.21 | patient also |
| | | | | | has Gaucher |
| MLD patient 6 | | 14 month | 0.0055 ± 0.0031 | 0.14 | |
| MLD patient 7 | c.459 + 1G > A//c.1277C > T | 15 month | 0.0064 ± 0.0002 | 0.17 | |
| MLD patient 8 | | 18 month | 0.0198 ± 0.0018 | 0.52 | |
| MLD patient 9 | | 18 month | 0.0056 ± 0.0011 | 0.15 | |
| MLD patient 10 | | 18 month | 0.0052 ± 0.0022 | 0.14 | |
| MLD patient 11 | p.E198A199del//c.848 + lG > A | 25 month | 0.0078 ± 0.0018 | 0.20 | |
| MLD patient 12 | c.1175G > A//c.442G > A | 29 month | 0.0128 ± 0.0003 | 0.33 | |
| MLD patient 13 | c.136T > C//c.115G > A | 30 month | 0.1533 ± 0.0108 | 3.99 | |
| MLD patient 14 | c.1144G > A//c.459 + 1G > A | 35 month | 0.2212 ± 0.0142 | 5.75 | |
| MLD patient 15 | c.459 + 1G > A//c.1277C > T | 60 month | 0.0078 ± 0.0011 | 0.20 | |
| MLD patient 16 | c.465 + 1G > A//c.869G > A | 60 month | 0.0139 ± 0.0005 | 0.36 | |
| MLD patient 17 | c.931G > A//c.931G > A | early juvenile | 0.0040 ± 0.0005 | 0.10 | |
| MLD patient 18 | c.465 + 1 G > A//c.869 G > A | early juvenile | 0.0057 ± 0.0006 | 0.15 | |
| MLD patient 19 | c.1136C > T/c.1136C > T | early juvenile | 0.0063 ± 0.0013 | 0.16 | |
| MLD patient 20 | | 100 month | 0.0068 ± 0.0018 | 0.18 | |
| MLD patient 21 | c.459 + 1G > A//c.1277C > T | | 0.008 ± 0.0004 | 0.21 | |
| MLD patient 22 | c.459 + 1G > A//c.1277C > T | | 0.0067 ± 0.0002 | 0.17 | |
| MSD patient 1 | | 18 month | 0.0046 ± 0.0015 | 0.12 | |
| Adult control | | | 3.8438 ± 0.0971 | | |

¹ARSA activity was measured in triplicate on three aliquots from the same batch of leukocyte lysate.

ARSA activity in leukocyte lysate was expressed as nmol/h/mg protein or as a percentage of the activity measured in the leukocyte lysate from a single healthy adult donor. MLD patient 21 and 22 were identified through their affected siblings, and were asymptomatic at the time of sampling, therefore their ages of disease onset were unavailable. MLD patient 5 also had Type I Gaucher disease, therefore the MLD variants were not the only contributing factor to the age of onset. The most severe MLD patients (late-infantile onset) in our cohort displayed symptoms between 12-35 months and had residual ARSA activity in the range of 0.03-5.8% compared to the healthy adult. MLD patient 13 and 14, two late infantile patients, displayed higher residual ARSA activity (4.0% and 5.8%) than the rest of the late-infantile patient cohort. Four MLD patients with juvenile onset (MLD patient 15-19) had residual ARSA activity below 0.4% when compared to the healthy adult. One MLD patient (MLD patient 20) showed initial symptoms at 100 months and displayed 0.18% residual ARSA activity. Therefore, with this limited dataset, there appeared to be no correlation between residual ARSA activity in leukocyte lysate and the age of disease onset. The patient genotypes were not predictive of the age of disease onset as well. For example, MLD patient 7 and 15 had the same genotype (c.459+1G>A//c.1277C>T) and similar residual leukocyte ARSA activity (0.20% and 0.17% of the healthy adult), yet their age of onset was 45 months apart.

An additional multiplexed assay was used to measure the activity of iduronate-2-sulfatase (I2S), N-acetylgalactosamine-6-sulfatase (GALNS), N-acetylgalactosamine-4-sulfatase (ARSB), α-N-acetylglucosaminidase (NAGLU), and lysosomal β-glucuronidase (GUSB) in these leukocyte lysates containing 2 μg protein using a previously published protocol (Liu, Y.; Yi, F.; Kumar, A. B.; Chennamaneni, N. K.; Hong, X.; Scott, C. R.; Gelb, M. H.; Turecek, F., Multiplex Tandem Mass Spectrometry Enzymatic Activity Assay for Newborn Screening of the Mucopolysaccharidoses and Type 2 Neuronal Ceroid Lipofuscinosis. Clin Chem 2017). All MLD patients had activities above 50% when compared to the healthy adult donor, whereas the MSD patient had sulfatase (I2S, GALNS and ARSB) activities below 2%, consistent with the diagnosis of MSD.

Application of ARSA assay to DBS extracts purified by immune-precipitation. Initial attempts to detect ARSA enzymatic activity in DBS were unsuccessful. When the leukocyte lysate was substituted with a 3 mm DBS punch in the ARSA assay, no ARSA activity could be detected. Moreover, no ARSA activity was observed when leukocyte lysate was co-incubated with a DBS punch, whereas activity was seen when the leukocyte was incubated with a filter paper punch lacking blood matrix (data not shown). This indicated the presence of some ARSA inhibitor(s) in whole blood. Addition of 5 mM Ce(Acetate)₃ or Pd(Acetate)₂ to the assay buffer did not rescue ARSA activity in DBS (data not shown), suggesting that the inhibitor(s) in whole blood were not inorganic sulfate or phosphate ions alone.

In an earlier study, Tan et al. were able to detect ARSA enzymatic activity in DBS after immuno-precipitating the ARSA protein in the DBS extract and the activity was then measured by the generic fluorogenic substrate for sulfatases, 4-methylumbelliferyl sulfate (Tan, M. A.; Dean, C. J.; Hopwood, J. J.; Meikle, P. J., Diagnosis of metachromatic leukodystrophy by immune quantification of arylsulphatase A protein and activity in dried blood spots. Clinical Chemistry 2008, 54 (11), 1925-1927). In one embodiment of the assays of the invention, the blood extract was purified from DBS by immuno-precipitation, and the ARSA activity was readily detected by LC-MS/MS using sulfatide as substrate (FIG. 4). This is consistent with the previous report and indicated that the ARSA inhibitor(s) from whole blood were removed during the immuno-precipitation. However, six other commercially available anti-ARSA serums were tested, including one polyclonal and five monoclonal, but no ARSA activity could be recovered after immuno-precipitation.

FIG. 4 shows the ARSA enzymatic activity in DBS from 4 MLD patients (median: 0.007 μM/h, range: 0.005-0.011 μM/h), 1 MSD patient (0.087 μM/h), and 7 healthy adults (0.63 μM/h, range: 0.39-1.30 μM/h) after the DBS extract was purified by immune-precipitation. All MLD patients had barely detectable ARSA activity, and the MSD patient displayed 14% ARSA activity when compared to the mean activity of the healthy adults, indicating these patients had essentially no residual ARSA activity.

The ARSA DBS assay with immuno-precipitation was used to investigate the stability of this enzyme in DBS. When stored at room temperature with desiccants, ARSA activity dropped to 95% after 7 days and to 60% after 14 days. Interestingly, additional activity loss was minimal over the next 100 days. ARSA activity remained stable in DBS when stored at 4° C. or −20° C. with desiccants for over 3 months. These results suggested that DBS should not be kept at room temperature over an extended period.

Application of ARSA assay to DBS extracts purified by size-exclusion chromatography. Given that the ARSA activity in DBS could only be measured after immuno-precipitation using a commercially available polyclonal antibody, alternative purification methods were sought that do not rely on a reagent in limited quantity. Initial attempts were based on ion exchange chromatography as it was previously reported for the purification of recombinantly expressed ARSA. However, no ARSA activity was recovered in the DBS extract purified by either cation or anion exchange chromatography (data not shown), probably because NaCl at high concentration is a strong inhibitor of ARSA. Size-exclusion chromatography was pursued to purify ARSA from the DBS matrix. ARSA activity was readily detected after DBS extract was purified with resins of various MW cutoff. The low-cost Sephadex G-25 resin with a MW cutoff of 5 k Da gave optimal results and was used in all the following studies.

The extraction protocol of ARSA from the DBS punch was then optimized. It was found that the color of the extract from poorly stored DBS punch was pale compared to extract from DBS stored under optimal conditions, and the ARSA activity measured in such pale extract was substantially lower, suggesting that not all of the protein was extracted from the punch. To improve the protein recovery, different extraction buffers were tested, including ARSA buffer, ammonium hydroxide in water and ammonium hydroxide in ARSA buffer, with a 50% increase in ARSA recovery when 0.5% ammonium hydroxide was in the ARSA buffer. The use of ammonium hydroxide was based on an earlier study showing improved extraction of proteins from DBS using this additive (Borremans, B., Ammonium improves elution of fixed dried blood spots without affecting immunofluorescence assay quality. Trop Med Int Health 2014, 19 (4), 413-6). Further optimization of the amount of ammonium hydroxide in the ARSA buffer gave the optimal DBS extraction buffer (0.8% ammonium hydroxide in ARSA buffer). The extraction time was optimized, and 4 hours at room temperature was chosen for optimal extraction. The amount of resin used per assay was varied for the size-exclusion chromatography and found that 60 mg dry resin per assay gave better consistency compared to 40 mg per assay, although at a cost of 20% loss in activity.

With the final ARSA DBS assay in hand, the variation in ARSA activity was validated across a series of Quality Control DBS for lysosomal storage disorders from the CDC (base pool (0.023±0.003 μM/h), low (0.048±0.007 μM/h), medium (0.21±0.02 μ/h), and high (0.37±0.02 μM/h) standards) (FIG. 5). These QC samples were prepared by mixing various amounts of unprocessed cord blood with base pool blood (prepared from leukocyte-reduced blood and heat-inactivated, charcoal-stripped serum) (De Jesus, V. R.; Zhang, X. K.; Keutzer, J.; Bodamer, O. A.; Muhl, A.; Orsini, J. J.; Caggana, M.; Vogt, R. F.; Hannon, W. H., Development and evaluation of quality control dried blood spot materials in newborn screening for lysosomal storage disorders. Clin Chem 2009, 55 (1), 158-64). FIG. 5 demonstrates the assay had good linear response (R²>0.99) and good reproducibility (<15% CV) with 20 replicates at each point. It should be noted that there was finite ARSA activity in the base pool sample (non-zero intercept of FIG. 5) after blank subtraction, showing that not all of the ARSA enzyme was depleted in the base pool DBS.

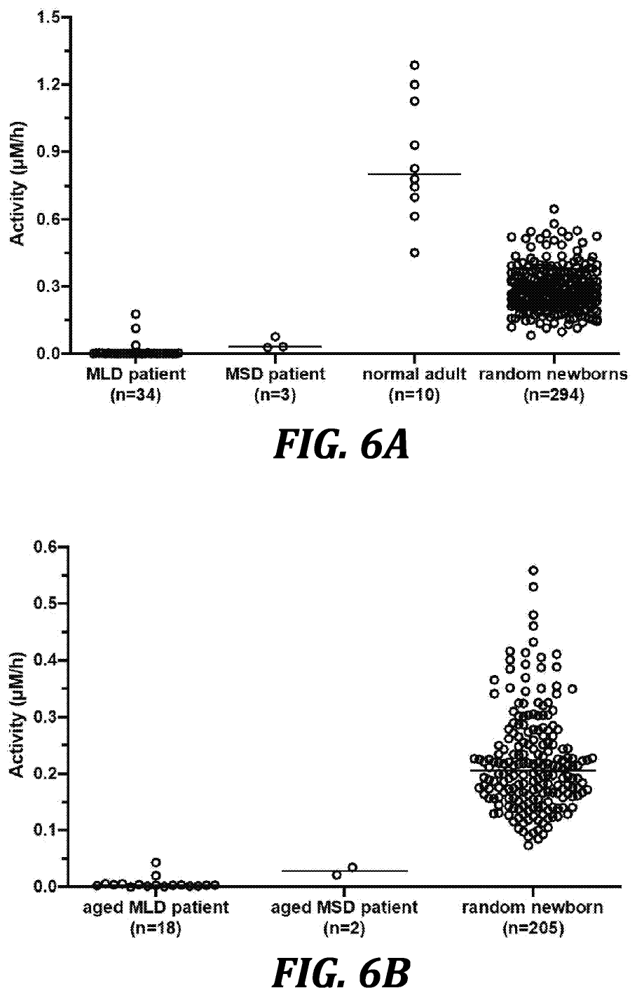

Having fully optimized and validated the ARSA DBS assay, ARSA activity in DBS was measured from 34 MLD patients (median: 0.0015 μM/h, range: 0-0.18 μM/h), 3 MSD patients (median: 0.032 μM/h, range: 0.028-0.076 μM/h), 10 healthy adults (median: 0.80 μM/h, range: 0.45-1.3 μM/h), and 294 presumed random newborns (median: 0.27 μM/h, range: 0.082-0.65 μM/h) (FIG. 6A). The two MLD patients that had the highest ARSA DBS activity (0.11 and 0.18 μM/h) were MLD patient 13 and 14 (Table 1), respectively. It should be noted that the random newborn DBS were stored for 1-2 months at room temperature. Presumably about 40% of the ARSA activity had been lost in these samples (based on DBS stability data), thus the range of activities in random newborns showed in FIG. 6A was lower than what would be expected in fresh newborn DBS. The patients and normal adult DBS were stored at −20° C. shortly after DBS collection with presumably minimal loss of ARSA activity. Because it was extremely hard to obtain large amount of fresh newborn DBS, an aging experiment was carried out as a compromise. When fresh DBS from 18 MLD (including DBS from MLD patient 13 and 14) and 2 MSD patients were aged at room temperature for 1 month, over 50% of the residual ARSA activity in DBS from MLD patient 13 and 14 was lost. The result demonstrated that patients can be completely distinguished from the normal (random newborns) based on the ARSA DBS activity if they had similar storage condition (FIG. 6B).

As noted above, Han et al. reported an ARSA leukocytes assay that used non-deuterated C18:0-sulfatide as substrate and commercially available d₃₅-C18:0-galactosyl-ceramide as internal standard (Han, M.; Jun, S. H.; Song, S. H.; Park, H. D.; Park, K. U.; Song, J., Ultra-performance liquid chromatography-tandem mass spectrometry measurement of leukocyte arylsulfatase A activity using a natural substrate. Ann Lab Med 2015, 35 (1), 165-8). In the assays described herein, deuterium labeled sulfatide was used as the enzymatic substrate so that the deuterated product can be differentiated from the endogenous galactosyl-ceramide.

Moreover, because the product, d₃₅-C18:0-galactosyl-ceramide, does not co-elute with the enzymatic product due to the 35 deuterium labels, the assays described herein use d₇-C18:0-galactosyl-ceramide, which co-elutes with the enzymatic product as the internal standard. Co-elution of the analyte and its internal standard is important for LC-MS/MS analysis as there could be different matrix effects on analytes eluting at different retention time. Surprisingly, the ARSA activity in leukocyte lysate from healthy adults reported by Han et al. were approximately 100-fold higher than those measured by the assays of the present invention. The enzymatic product measured by Han et al. may have been a combination of enzymatic breakdown of sulfatide and the galactosyl-ceramide present in the sample and the activities reported by Han et al. were in general 30 to 100-fold higher than the activities of other lysosomal enzymes measured in leukocytes.

The LC-MS/MS ARSA assays of the present invention are more accurate and precise than the traditional colorimetric and fluorometric ARSA assays, where generic artificial sulfatase substrates are used. Because these generic substrates are not specific to ARSA, these assays either required two assays performed in parallel, one with an ARSA specific inhibitor and one without, or required the removal of isoenzymes by ion-exchange chromatography. Inhibition of ARSA is only partial in these earlier methods as the inhibitors used are neither highly potent, nor ARSA-specific, which compromises the reliability of the assay, especially at the lower end. Usage of assay buffer that favors ARSA activity has been reported, but it is not clear if the residual activity is due to ARSA, or other off-target sulfatases, or both. This is a serious concern as proper evaluation of trace amounts of residual ARSA activity is crucial when diagnosing potential patients. Recently, a new spectrophotometric ARSA assay was developed using sulfatide as substrate (Morena, F.; di Girolamo, I.; Emiliani, C.; Gritti, A.; Biffi, A.; Martino, S., A new analytical bench assay for the determination of arylsulfatase a activity toward galactosyl-3-sulfate ceramide: implication for metachromatic leukodystrophy diagnosis. Anal Chem 2014, 86 (1), 473-81). However, this assay quantified the enzymatic activity by measuring the decrease of substrate instead of the formation of product, therefore it will not be accurate to measure trace amounts of residual ARSA activity as well. It is also possible to use generic sulfatase substrates to selectively assay ARSA after immuno-precipitation purification, but this requires a polyclonal antiserum that is available in limited quantities, and also relies on the assumption that no off-target sulfatases are being pulled down during the process. The LC-MS/MS-based ARSA assay of the invention is highly ARSA-specific due to the usage of the natural substrate and was highly precise and sensitive as trace amounts of residual activity could be detected with statistical significance (FIG. 3). It should be noted that the methods described in this study are for research only and do not meet The Clinical Laboratory Improvement Amendments (CLIA) validation guidelines for the development of laboratory developed tests (LDTs). Furthermore, studies of a larger cohort of patients and normal controls are needed to define a reference range. The fluorometric and LC-MS/MS assays are both adequate for diagnostic purposes, where measurement of nominally low enzymatic activity is potentially sufficient for diagnosis when the patient exhibits symptoms that are characteristic of the disease. However, due to its high performance, the ARSA leukocyte assay of the invention is beneficial for evaluating potential patients who are identified by NBS and may be at risk to develop MLD but are so far asymptomatic. This is especially important in the case of MLD where there is a high frequency of pseudodeficiency variants.

The ARSA assays of the invention are able to detect ARSA activity in DBS only if the enzyme is purified or partially purified from the matrix. Immunoprecipitation of ARSA has useful in this context, but to date a monoclonal antibody that works in the enzymatic assay remains unknown. Reliance on a commercially available polyclonal anti-ARSA antiserum is problematic for long-term sustainability of the assay. Fortunately, in one embodiment of the assays described herein, a simple size-exclusion procedure was found to be sufficient to remove the inhibitor(s) and make ARSA detectable in the DBS matrix. Furthermore, the protocol is simple and inexpensive to carry out. With automation, it is also appropriate for high throughput screening including NBS.

The ARSA enzymatic activity assay in DBS of the invention is implemented as the second-tier test for a large-scale de-identified research study for the NBS of MLD, where the ARSA enzymatic assay is performed on samples with abnormal sulfatide results, as described below. With this two-tier algorithm, the false positive rate is largely reduced and so is the accompanying anxiety to families in a real-world scenario. It should be noted that the ARSA activity in newborns reported here cannot be used as a normal range as these DBS were stored at room temperature for 1-2 months prior to analysis, and ARSA is known to be unstable under this storage condition.

A second advantage of having an ARSA enzymatic assay using DBS is that the shipment of EDTA-whole blood to diagnostic laboratories often results in samples that do not allow sufficient leukocytes to be isolated.

In summary, the present invention provides an ARSA leukocyte and a DBS assay by LC-MS/MS. Both assays used deuterated natural sulfatide as substrate, therefore are highly specific to ARSA. This is essential for accurately diagnosing MLD and MSD patients.

### Newborn Screening For Metachromatic Leukodystrophy: Two-Tier Screening

Newborn screening for metachromatic leukodystrophy is essential for early diagnosis and therefore optimal therapeutic outcomes. The feasibility of screening MLD using dried blood spots (DBS) from de-identified newborns was assessed. To minimize the false-positive rate, a two-tier screening algorithm was used. The primary test was to quantify C16:0-sulfatide in DBS by high throughput ultra-performance liquid-chromatography tandem mass spectrometry (UPLC-MS/MS). The screening cut-off was established based on the results from 15 MLD newborns to achieve 100% sensitivity. The secondary test was to measure the ARSA activity in DBS from newborns with abnormal C16:0-sulfatide levels. Only newborns that displayed both abnormal C16:0-sulfatide abundance and ARSA activity were considered screen positives. A total of 27,335 newborns were screened using this algorithm, and 2 high-risk cases were identified. ARSA gene sequencing identified these two high-risk subjects to be an MLD-affected patient and a carrier indicating that the screening method was highly specific.

### UPLC-MS/MS Analysis of Sulfatide in DBS

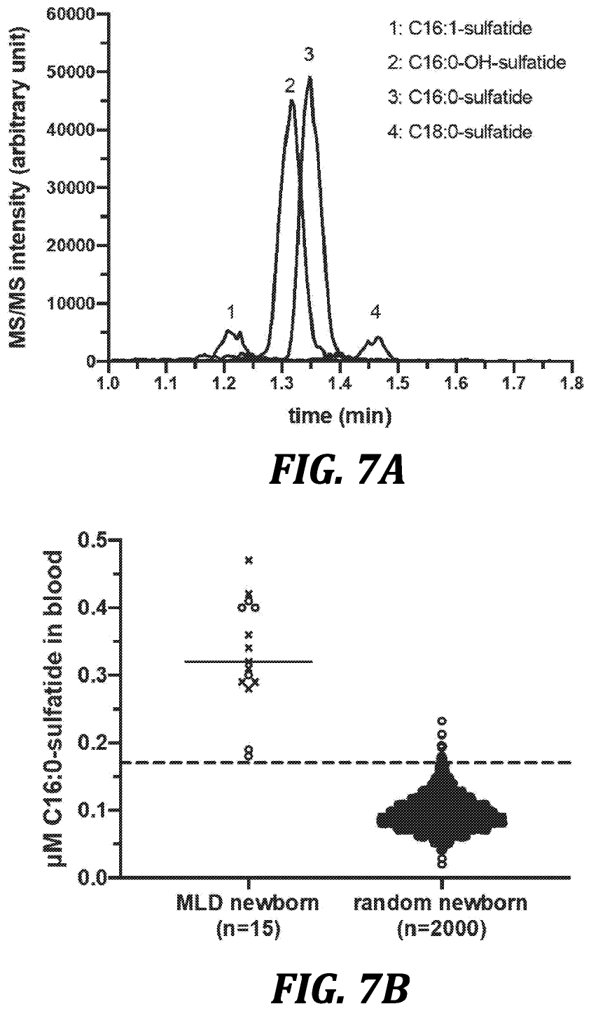

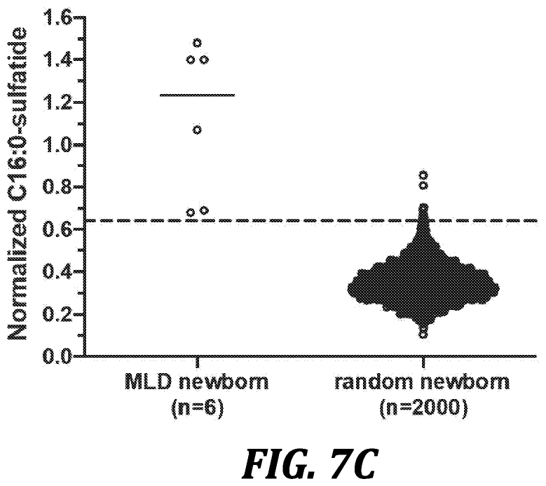

At least four sulfatide species were elevated in DBS from MLD patients compared to healthy controls. C16:0- and C16:0-OH-sulfatide (named according to the fatty acyl group attached to the sphingosine backbone) account for more than 85% of the sulfatides in blood, whereas C16:1-OH- and C18:0-sulfatide account for the remainder. Therefore, a study of the newborn screening of MLD was initiated by monitoring the total amount of these four sulfatide species in DBS using high throughput UPLC-MS/MS. FIG. 7A shows a typical UPLC-MS/MS chromatogram of C16:1-OH, C16:0-OH, C16:0, and C18:0-sulfatide in DBS from a random newborn. The sample injection-to-injection time was 2.5 minutes, allowing more than 500 samples to be analyzed per day per instrument.

### Sulfatide Analysis in DBS From MLD Newborns

DBS from a total of 15 MLD newborns were acquired throughout the study (obtained from newborn screening laboratory repositories). Individual molecular sulfatide species levels in DBS and age of onset information for these 15 newborn MLD DBS were determined. Among these 15 MLD newborns, two patients with juvenile MLD displayed substantially lower sulfatide level compared to the rest of the cohort. The sulfatide assay was repeated on punches from a second set of DBS from these patients with the same results. An ARSA protein abundance assay and an ARSA enzymatic activity assay were also performed, with essentially no ARSA protein and ARSA activity detected. Together, these results suggest that the correct archived DBS were pulled out of storage. These archived DBS were stored at −20° C. Sulfatides should be relatively stable under this storage condition for at least 1 year based on our experience with adult DBS. Elevation in blood sulfatides was observed in an MLD newborn with late-infantile onset caused by saposin B deficiency, which was consistent with reports.

### Analysis of Total Sulfatide in DBS

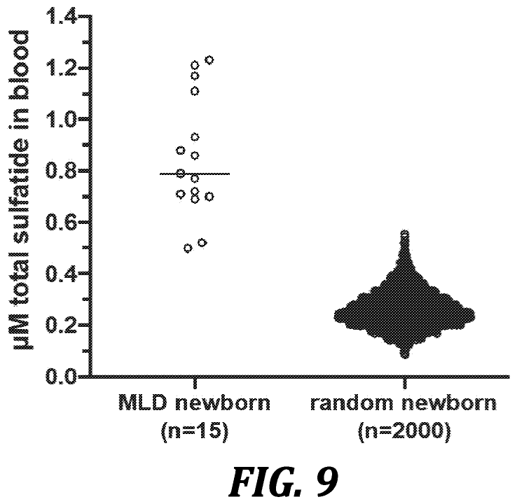

FIG. 9 shows the total sulfatide abundance in 15 MLD newborns (median: 0.79 μM, range: 0.50-1.23 μM) and 2,000 random newborns (median: 0.25 μM, range:

0.085-0.56 μM).

### Analysis of C16:0-Sulfatide in DBS

FIG. 7B displays the C16:0-sulfatide concentration in DBS from 15 MLD newborns (median: 0.32 μM, range: 0.18-0.47 μM) and 2,000 random newborns (median: 0.094 μM, range: 0.020-0.23 μM). A distribution overlap between the diseased and random newborns was observed (FIG. 7B). 0.17 μM C16:0-sulfatide was used as the screening cut-off to achieve 100% assay sensitivity.

To further harmonize the results, for every 96-well plate of newborn DBS, a separate UPLC-MS/MS run was carried out by injecting an authentic C16:0-sulfatide standard along with d₅-C16:0 internal standard. The C16:0-sulfatide/internal standard ion ratio measured for each newborn was divided by the average ratio measured for 5 injections of the C16:0-sulfatide standard (normalization method). The C16:0-sulfatide level in groups of 500 newborns collected over 5 months with or without this normalization. Results with normalization were more consistent between groups, suggesting that the variations were mostly introduced from internal standard batch-to-batch differences as well as fluctuations of the mass spectrometer, which could be accounted for by the normalization to the external calibrator.

The approach for the screening of MLD was to quantify C16:0-sulfatide in DBS using d₅-C16:0-sulfatide as the internal standard and then to normalize to the external calibrator (C16:0-sulfatide standard processed at the same time as the DBS). FIG. 7C displays the normalized C16:0-sulfatide level in DBS from 6 MLD newborns (median: 1.24, range: 0.68-1.48) and 2,000 random newborns (median: 0.34, range: 0.11-0.86). Normalized C16:0-sulfatide results from the 2 MLD newborns displaying the lowest sulfatide abundance, which allowed for defining the cut-off to achieve 100% sensitivity. The screening cut-off was set at 0.64 after the normalization, which corresponds to 0.17 μM C16:0-sulfatide in blood (FIG. 7C).

A total of 27,335 random newborns were screened using this strategy, of which 195 (0.71%) had normalized C16:0-sulfatide level above the cut-off (see Table 2).

TABLE 2
Summary of results using different screening strategies.

| | | | Newborns | Newborns |
|---|---|---|---|---|
| | | | with abnormal | with abnormal |
| Screening | Newborns | Screening | 1^{st} tier | 2^{nd} tier |
| method | screened | cut-off | results | results |
| C16:0- | 27.335 | 0.64 after | 195 | 2 out of 122^{a} |
| sulfatide | | normalization | | |
| ARSA | 2,287 | 20% daily | 3 | 0 out of 3^{b} |
| activity | | mean activity | | |

^{a}The 2^{nd} tier test was to measure ARSA activity in DBS. Only 122 out of the 195 newborns with abnormal C16:0-sulfaitde results were submitted for the ARSA activity assay. The remaining were not processed for ARSA activity as the DBS were too old (>3 months at room temperature) for the test.
^{b}The 2^{nd} tier test was to measure C16:0-sulfatide in DBS

### Implementation of ARSA DBS Activity Assay as a Second-Tier Test

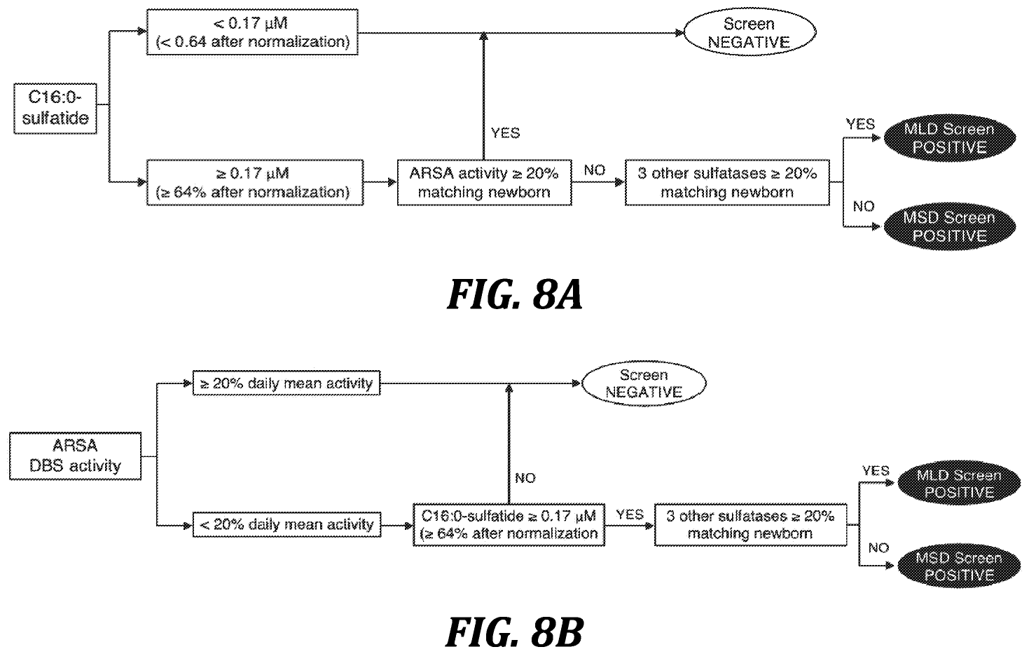

A two-tier screening algorithm was adopted given that the C16:0-sulfatide analysis resulted in a 0.71% screen-positive rate (FIG. 8A). An ARSA DBS enzymatic activity assay was implemented as the second-tier test using an additional 3-mm punch from the same DBS that was used for sulfatide analysis. Because ARSA is known to be unstable in DBS at room temperature, ARSA activity was also measured in “matching newborns” with normal sulfatide levels and similar storage conditions to define a reference range. For newborns with abnormal C16:0-sulfatide levels, those with normal ARSA activity were considered to be screen negatives, whereas those with abnormally low ARSA activity were deemed as screen positives (FIG. 8B). In the latter case, activities of three additional sulfatases (I2S, GALNS, and ARSB) were measured to further distinguish MLD screen positives from MSD screen positives (FIG. 8B).

Among those 195 newborns with C16:0-sulfatide abundance above 0.64 after normalization, 122 were submitted for ARSA activity assay. The rest were not tested for ARSA activity as the DBS were too old (more than 3 months at room temperature). All but two newborns with abnormal C16:0-sulfatide level had ARSA activity above 20% relative to their matching newborns, showing that they were all false positives identified by the sulfatide assay (Table 2). Between the two newborns with abnormal sulfatide and ARSA activity levels, subject 24 displayed C16:0-sulfatide at 0.86 after normalization and ARSA activity of 0% of the matching newborns, while subject 128 displayed C16:0-sulfatide at 0.72 after normalization and ARSA activity of 8% of the matching newborns. Activities of I2S, GALNS and ARSB were only measured in subject 128, with the activities all above 20% of the mean activities of its matching newborns, ruling out the possibility of MSD. Even though there were no I2S, GALNS and ARSB activity data on subject 24, reserved DBS punches from these two newborns were both submitted for ARSA gene sequencing because MSD is much more rare than MLD.

### ARSA Genomic Variant Analysis

ARSA whole-exome sequencing was performed on 5 DBS samples for variant discovery. The samples consisted of 2 MLD screen positives (subject 24 and 128) and 3 MLD screen negatives (elevated sulfatides but high ARSA enzymatic activity, subject 9, 11, and 23). Three variants, including 1 exonic (c.1178C>G) and 2 intronic (c.1210+20C>G and c.1108-32C>T), were found across all 5 samples. c.1178C>G is a known pseudodeficiency variant. c.1210+20C>G is classified as benign in ClinVar. While c.1108-32C>T is not reported in ClinVar, it has higher than expected population allele frequencies and was therefore not considered to be pathogenic (0.7979, gnomAD). Additionally, 1 screen positive and 2 screen negative samples had the common pseudodeficiency variant c.1055A>G (p.Asn352Ser). Pathogenic variants were only discovered in the 2 screen positive samples. The status of “screen negative” of subject 9, 11, and 23 were confirmed by ARSA sequencing.

Subject 24 was heterozygous for the pathogenic variants c.1283C>T and c.1292A>C. Although ClinVar does not have an interpretation for c.1292A>C (previously known as c. 1286A>C), this variant has been listed as pathogenic in multiple reports. This subject was also heterozygous for the common pseudodeficiency variant, c.1178C>G. Therefore, subject 24 was interpreted as an MLD-affected patient.

Subject 128 was heterozygous for c.1174C>T. ClinVar has conflicting interpretations of pathogenicity (pathogenic or uncertain significance) for c.1174C>T (previously known as c. 1168C>T), but it is deemed as pathogenic according to multiple literature reports. This subject is also heterozygous and homozygous for the pseudodeficiency variants c.1178C>G and c.1055A>G, respectively. Taken together, subject 128 was interpreted as a carrier.

### ARSA DBS Activity Assay as the First-Tier Screening Test For MLD

Because the sulfatide assay resulted in a high false-positive rate (0.71%), whether the ARSA DBS activity assay could suffice as the primary screening test was explored. Shown in FIG. 8B is the screening algorithm, where the C16:0-sulfatide DBS assay was implemented as the secondary test. The selection of the 20% daily mean activity cut-off was based on previously reported data.

ARSA activity in DBS from 2,287 de-identified newborns were measured by the ARSA assay as described herein. Three out of the 2,287 newborns (0.13%) had ARSA activity below the cut-off, all of which were considered screen negatives based on the C16:0-sulfatide results (Table 2). Results from the 2,287 newborns and the three subjects with low ARSA activity are summarized in Table 3.

TABLE 3
Results from the 3 newborns with ARSA activity below
the cut-off (20% of daily mean activity) and 2,287
newborns screened based on the ARSA DBS activity.

| | ARSA | % to | C16:0- | Normalized |
|---|---|---|---|---|
| | activity | daily | sulfatide | C16:0- |
| | (μM/h) | mean | (μM) | sulfatide |
| subject 1 | 0.035 | 19 | 0.092 | 0.330 |
| subject 2 | 0.012 | 13 | 0.162 | 0.586 |
| subject 3 | 0.004 | 2 | 0.128 | 0.463 |
| random | 0.157 | n/a | n/a | n/a |
| newborns^{a} | (0.041-0.430, | | | |
| | n = 2,287) | | | |

^{a}Data for random newborns are expressed in median (1-99 percentile, number of newborns).

Two screen positives were identified out of 27,335 newborns, among which one is an MLD-affected patient while the other is a carrier. The false-positive rate (0.0037%) is exceptionally low compared to the other active newborn screening programs, highlighting the specificity of the two-tier screening algorithm.

To date, lysosomal storage diseases that are due to malfunctioning enzymes are typically screened using functional assays; but ARSA DBS activity assay was not available until recently. This screening strategy was further discredited due to two major concerns: (1) the potential false-positive problems caused by high prevalence of pseudodeficiency variants; and (2) the thermal instability of ARSA in DBS. Nonetheless, given the high false-positive rate (0.71%) of the sulfatide assay; ARSA activity assay may suffice as the primary test while using the C16:0-sulfatide assay as a secondary test (FIG. 8B). This is supported by the lower false-positive rate (0.13%) from the small-scale study on 2,287 newborns (Table 2). 50% of the ARSA activity remained when the DBS was stored under extreme conditions for three days. Because DBS should be delivered to the screening laboratories within 3 days of sample collection, the ARSA thermal instability may not be a major issue; though it remains to be seen how problematic this will be in warmer parts of the world. Currently the ARSA assay is more complex than the sulfatide assay; nevertheless, with automation the ARSA activity and sulfatide assays can be carried out by a single laboratory person and multiplexed with other assays.

In conclusion, the large-scale study described herein demonstrates that newborn screening for MLD is feasible with an exceptionally low false-positive rate (0.0037%) if a two-tier screening strategy is adopted: newborns screened based on the abundance of blood sulfatide and ARSA enzymatic activity be measured on those with abnormal sulfatide results. This two-tier screening approach is crucial for the balance between assay sensitivity and specificity. A total of 27,335 newborns were screened with this approach, and only 2 of which were considered screen positives. ARSA gene sequencing identified one as an MLD patient and the other as carrier, demonstrating this two-tier strategy is highly specific. Alternatively, it may also be possible to screen for MLD using the ARSA activity assay as the primary test, and the sulfatide assay as the secondary test. Implementation of these two-tier screening strategies will be useful in identifying patients who will then benefit from upcoming novel therapies.

### ARSA Assay: Materials and Methods

Whole blood from a healthy adult donor and DBS from 10 healthy adults were collected with consent and were used as positive controls. Whole blood from MLD and MSD patients were obtained through the Myelin Disorders Biorepository Project at the Children's Hospital of Philadelphia, the MLD Foundation, Duke University, the Children's Hospital of Pittsburgh and the San Raffaele Telethon Institute for Gene Therapy (Milan, Italy) with IRB approvals. DBS was prepared by spotting the whole blood onto 903 protein saver cards (GE Healthcare Life Sciences). Additional DBS from MLD patients were obtained through Meyer Children's Hospital (Florence, Italy). All patients were diagnosed based on clinical, biochemical and genetic evidence of the disease. De-identified newborn DBS, which were previously stored at room temperature for 30-60 days, were acquired through the Washington State Department of Health with the approval from the Washington State Institutional Review Board. Quality Control DBS for lysosomal storage disorders was acquired from the Centers for Disease Control and Prevention (CDC).

d₃-C18:0-Sulfatide was purchased from Matreya, LLC (Cat. 1536). d₇-C18:0-Galactosyl-ceramide was synthesized and was quantified by quantitative ¹H-NMR as described below in Example 1. Stock solutions of sulfatide and galactosyl-ceramide were prepared in 2/1 chloroform/methanol (v/v) and were stored in glass vials with Teflon-septum screw caps at −20° C. ARSA-containing lymphoblasts (GM14603) and ARSA-lacking lymphoblasts (GM23097) were obtained from the Coriell Institute Cell Repository and were cultured as per the vendor's instructions.

Preparation of leukocytes and BCA protein assay. Venous blood (at least 0.5 mL) was collected into a K₂EDTA tube and mixed by inversion to distribute the anti-coagulant. The blood was stored at 4° C. prior to overnight shipment in insulated boxes with frozen gel packs. Leukocytes were prepared within 24 hours of blood collection as previously described (Lin, N.; Huang, J.; Violante, S.; Orsini, J. J.; Caggana, M.; Hughes, E. E.; Stevens, C.; DiAntonio, L.; Chieh Liao, H.; Hong, X.; Ghomashchi, F.; Babu Kumar, A.; Zhou, H.; Kornreich, R.; Wasserstein, M.; Gelb, M. H.; Yu, C., Liquid Chromatography-Tandem Mass Spectrometry Assay of Leukocyte Acid alpha-Glucosidase for Post-Newborn Screening Evaluation of Pompe Disease. Clin Chem 2017, 63 (4), 842-851). Leukocytes processed from whole blood collected in heparin tubes was also suitable for the assay but required additional validations. Supernatant and leukocyte suspension were then stored at −80° C. Phosphate-buffered saline should be avoided as inorganic phosphate strongly inhibits ARSA.

Cells were stored at −80° C. for at least 16 hours prior to lysis by thawing on ice. The lysate was centrifuged at 10,000 g for 5 min at room temperature, and the supernatant was transferred to a new tube. The concentration of protein was determined by the Microplate BCA kit (ThermoFisher, Cat. 23252), using bovine serum albumin as the standard. Leukocyte lysates were typically assayed for protein concentration and ARSA activity right after thawing. However, multiple freeze-thaw cycle had minimal effect on the ARSA activity (FIG. S6b).

ARSA leukocyte assay. Assay cocktail consisted of 150 μM d₃-C18:0-sulfatide as substrate and 2 μM d₇-C18:0-galactosyl-ceramide as internal standard in ARSA assay buffer (80 mM sodium acetate (J.T. Baker, Cat. 3460-01), 2.0 g/L sodium taurodeoxycholate (Carbosyth, Cat. FS45995), pH 4.5±0.02). It was prepared by mixing proper amounts of substrate and internal standard stock solution, then removing the organic solvent with a centrifugal concentrator or a jet of oil-free air at room temperature and reconstituting the residual with assay buffer with a vortex mixer. Assay cocktail was typically prepared fresh, but it could also be stored frozen at −20° C.

Leukocyte protein concentration was adjusted to 0.2 μg/μL using 0.9% NaCl in water as diluent. The assay was set up using 10 μL of leukocyte lysate containing 2 μg protein and 30 μL of assay cocktail in a 96-well, polypropylene deep-well plate sealed with a silicone matt. The plate was centrifuged for 1 min at 3000 g to ensure that all liquid was at the bottom of each well. The plate was placed on an orbital shaker (400 rpm on a 3 mm shaking radius) at 37° C. for 16 hours. Reactions were quenched by addition of 300 μL of methanol, and the plate was centrifuged for 5 min at 3000 g. Supernatant (150 μL) was transferred to the autosampler plate followed by addition of 50 μL water per well, and the plate was placed in the cooled (8° C.) autosampler chamber of the LC-MS/MS instrument.

ARSA DBS assay after purification by immuno-precipitation. Polyclonal anti-ARSA antiserum (R&D System, Cat. AF2485) was immobilized on a high binding plate (PerkinElmer, Cat. 1244-550) at 1 μg anti-ARSA per well as follows. Anti-ARSA (100 μL of 10 μg/mL) in 0.2 M sodium phosphate, pH 6.8 was added in each well, and the plate was sealed with plastic film and incubated overnight on an orbital shaker at room temperature with gentle shaking. The solution was aspirated off, and 300 μL 0.9% NaCl in water was added per well, followed by aspiration. Blocking buffer (250 μL of 0.05 M Tris-HCl, pH 7.8, 0.9% NaCl, 0.05% NaN₃, 6.0% D-sorbitol, 1.0% bovine serum albumin (Sigma, Cat. A6003), 1.0 mM CaCl₂) was added, and the plate was sealed and incubated overnight on an orbital shaker at room temperature with gentle shaking. After aspiration, to each well was added 300 μL 0.9% NaCl, followed by aspiration. Plates were sealed with plastic film and stored in a sealed container with water for humidification. The coated plates can be stored at 4° C. for at least 6 months.

To each 3 mm DBS punch in a separate 96-well plate, 120 μL extraction buffer (0.1 M sodium acetate, 0.1% (w/v) bovine serum albumin, pH 5.0) was added. The plate was sealed and shaken on an orbital shaker at 37° C. for 1 hour. DBS eluent (100 μL) was transferred to an anti-ARSA coated well, and the plate was sealed and shaken on an orbital shaker at room temperature for 1 hour. The solution was removed by aspiration, and 300 μL of 20 mM sodium acetate, pH 5.0 was added to wash the well, followed by aspiration. To each well, 50 μL of assay cocktail (150 μM d₃-C18:0-sulfatide as substrate and 0.2 μM d₇-C18:0-galactosyl-ceramide as internal standard in assay buffer) was added. The plate was centrifuged for 1 min at 3000 g to ensure the bottom of each well was covered with liquid. The plate was sealed and placed on an orbital shaker (400 rpm on a 3 mm shaking radius) at 37° C. for 16 hours. The assay workup was the same as for the leukocyte assay.

ARSA DBS after purification by size-exclusion chromatography. To each 3 mm DBS punch in a 96-well plate, 50 μL extraction buffer (0.8% NH₄OH (Millipore, Cat. AX1303) in assay buffer) was added. The plate was sealed and shaken on an orbital shaker at room temperature for 4 hours. Sephadex G-25 resin (GE Healthcare Life Sciences, Cat. 17003201) was swollen in Milli-Q water (10 mL per g of dry resin) for 3 hours prior to use. In a 96-well fritted plate (ThermoFisher, Cat. 278011), 600 μL resin slurry (equivalent to 60 mg dry resin) was added per well using a pipette. Water was removed by centrifugation of the plate at 800 g for 1 min, and the resin was washed 3 times with 600 μL assay buffer, each time the assay buffer was removed by centrifugation at 800 g for 1 min. After the third wash, a clean 96-well plate was placed under the fritted plate as a receiver plate. DBS eluent (30 μL) was loaded on to the resin, followed by 15 μL of assay buffer. These liquids were added with the pipette tip against the side wall of the fritted well so that the resin pellet was not disturbed. The plate was centrifuged at 800 g for 1 min, and the eluate was collected. To the purified DBS eluate in the receiver plate, 10 μL assay cocktail (450 μM d₃-C18:0-sulfatide as substrate and 1 μM d₇-C18:0-galactosyl-ceramide as internal standard in assay buffer) was added. The plate was centrifuged for 1 min at 3000 g to ensure that all liquid was at the bottom of each well. The plate was sealed and placed on an orbital shaker (400 rpm on a 3 mm shaking radius) at 37° C. for 16 hours. The assay workup was the same as for the leukocyte assay.

UPLC-MS/MS analysis. UPLC-MS/MS analysis was carried out on a Waters Xevo TQ-S micro mass spectrometer coupled to a Waters AQUITY UPLC I-Class system using multiple reaction monitoring (MRM) in ESI positive mode. ESI and MRM settings are summarized in Tables 4 and 5 below.

TABLE 4
ESI source parameters

| Parameter | |
|---|---|
| Polarity | ES+ |
| Capillary Voltage (kV) | 3.5 |
| Source temperature (° C.) | 150 |
| Desolvation temperature (° C.) | 550 |
| Cone gas flow (L/hr) | 50 |
| Desolvation gas flow (L/hr) | 750 |
| Collision gas | Argon |

TABLE 5
MRM parameters

| | | Parent | Daughter | Dwell | Cone | Collision |
|---|---|---|---|---|---|---|
| | ESI | mass | mass | time | voltage | energy |
| Analyte | polarity | (m/z) | (m/z) | (s) | (V) | (V) |
| ARSA-P | + | 731.7 | 264.3 | 0.054 | 25 | 40 |
| ARSA-IS | + | 735.8 | 264.3 | 0.054 | 25 | 40 |
| ASRA-S | + | 811.5 | 264.3 | 0.054 | 25 | 40 |

Separation of the enzymatic substrate and product was achieved at a column temperature of 30° C. using an ACQUITY UPLC CSH Fluoro Phenyl column (1.7 μm, 2.1 mm×50 mm, Waters Corp., Cat. 186005351) connected to an ACQUITY UPLC CSH Fluoro Phenyl VanGuard Pre-column (1.7 μm, 2.1 mm×5 mm, Waters Corp., Cat. 186005358) at a flow rate of 0.7 mL/min. Mobile phase A was 50/50 (v/v) water/acetonitrile with 0.1% formic acid; mobile phase B was 50/50 (v/v) acetonitrile/isopropanol with 0.1% formic acid. The weak needle wash and the strong needle wash were 25/25/50 (v/v/v) methanol/isopropanol/water and 47.5/47.5/5 (v/v/v) methanol/isopropanol/water, respectively. The linear gradient was as followed: 0-0.5 min, 0% solvent B; 0.5-1.0 min, 0 to 90% solvent B; 1.0-1.5 min, 90 to 100% solvent B, 1.5-1.95 min, 100% solvent B, 1.95-2.0 min, 100 to 0% solvent B. All solvents were Optima Grade (Fisher Scientific).

Enzymatic activity calculation. ARSA activity in leukocyte (nmol/h/mg protein) was calculated by multiplying the ion ratio of ARSA product to ARSA internal standard (blank subtracted) by the nanomoles of internal standard added to the assay, then dividing by the incubation time (h) and the amount of leukocyte protein used (mg). ARSA activity in DBS (μM/h) was calculated by multiplying the ion ratio of ARSA product to ARSA internal standard (blank subtracted) by the μmoles of internal standard added to the assay, then dividing by the incubation time (h) and the volume of the blood (L), assuming each 3 mm DBS punch contained 3.2 μL blood. The product-to-internal standard MRM response ratio was assumed to be unity for an equimole mixture of product and internal standard.

### Synthesis of a Representative Isotopically-Labeled Substrate:

### d₇-18:0-galactosylceramide

The synthesis of a representative isotopically-labeled standard, d₇-18:0-galactosylceramide, is described and schematically illustrated below.

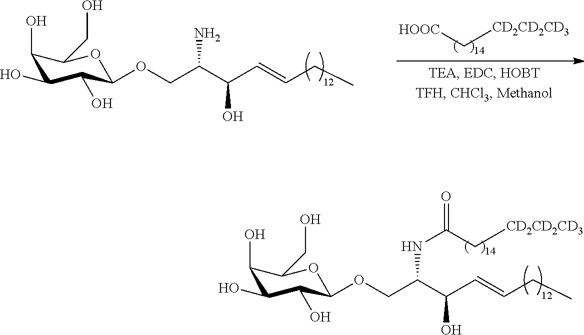

To a solution of stearic acid-d₇ (5 mg, 17.1 μmole, CDN Isotopes) in tetrahydrofuran (1 mL), triethylamine (6 μL), hydroxybenzotriazole (5 mg, 37.0 μmole) and 1-ethyl-3-(3-dimethylaminopropyl)carbodiimide (EDC, 6 mg, 31.3 μmole) were added successively at 0° C. To this mixture was added a 1 mL solution of psychosine (3.5 mg, 7.58 μmole, Avanti Polar Lipids) in chloroform/methanol (3:1). The mixture was warmed to room temperature and left to stir for 16 hours. The resulting mixture was dried and re-dissolved in isopropanol (2 mL), filtered and subjected to HPLC purification (reverse phase-C4 semi-preparative column, ACE HPLC columns Cat. No. ACE-133-1010) with 1:1 water/acetonitrile as solvent A and 1:4 acetonitrile/isopropanol as solvent B. The solvent program was 30% solvent B to 100% solvent B over 25 min at 4 mL/min. The fractions from HPLC with the desired fractions, as identified by LC-MS, were combined and concentrated to get the desired ARSA-IS (1.7 mg, 31%) as white solid. MS (ESI⁺) for [M+H]⁺; calculated: 735.6, found: 735.8.

Stock solutions of d₇-18:0-galactosylceramide were quantified by quantitative ¹H-NMR in CDCl₃/CD₃OD (4/1) solvent using N,N-dimethylformamide as an internal standard. The delay between NMR pulses was 10 sec to allow for full spin relaxation.

### Two-Tier MLD Screening Assay: Materials and Methods

DBS Samples

This study using DBS from de-identified newborns was approved by the Washington State Institutional Review Board. The DBS were shared by the Washington State Department of Health after being stored for 30-60 days at room temperature. A total of 15 archived DBS from MLD newborns (10 late-infantile and 5 juvenile onset) were acquired through the MLD foundation, the University of Pittsburgh, and the Meyer Children's Hospital (Florence, Italy). One of the 15 MLD newborns was diagnosed with saposin B deficiency (MLD newborn 6, Table 3). Additional adult DBS were prepared from K₂EDTA blood collected from a consenting healthy adult and were used as positive controls. Results from the random newborns and the 15 MLD newborns are summarized in Table 3.

Methods

Sulfatides from a 3-mm DBS punch were extracted with methanol containing the internal standard (d₅-C16:0-sulfatide). Methanol containing C16:0-sulfatide (external calibrators) were also included in each plate in triplicate and were processed at the same time as the DBS. After a 4-hour extraction, the sample was centrifuged, and the supernatant was analyzed by ultra-performance liquid chromatography tandem mass spectrometry (UPLC-MS/MS), where the analyte and its internal standard were detected by multiple reaction monitoring in ESI negative mode. The chromatographic peaks were integrated automatically using the TargetLynx software (Waters Corp.) and were inspected manually. The concentration of C16:0-sulfatide (μM) in blood was calculated by multiplying the analyte/internal standard ion ratio by the μmole of the internal standard added to the assay, then dividing by the volume of blood (L), assuming each 3 mm DBS punch contained 3.2 μL blood. Alternatively, the C16:0-sulfatide level in DBS was normalized to the external calibrator by dividing the analyte/internal standard ion ratio of C16:0-sulfatide of the DBS sample to the mean ion ratio of the triplicated calibrators in the plate. Newborns with normalized C16:0-sulfatide level above 0.64 were considered at risk of MLD and were subjected to a second-tier test.

A second 3-mm punch from newborns with sulfatide level above the screening cut-off, along with punches from 6-8 newborns with normal sulfatide level and similar storage condition (matching newborns) were submitted to the ARSA enzymatic assay as described herein. For newborns with sulfatide level above the cut-off (0.64 normalized C16:0-sulfatide) and ARSA activity below the cut-off (20% to the mean activity of the matching newborns), activities of iduronate-2-sulfatase (I2S), N-acetylgalactosamine-6-sulfatase (GALNS) and N-acetylgalactosamine-4-sulfatase (ARSB) were measured as previously described (Y. Liu et al., Multiplex tandem mass spectrometry enzymatic activity assay for newborn screening of the mucopolysaccharidoses and type 2 neuronal ceroid lipofuscinosis. Clinical Chemistry 63, 1118-1126 (2017)). Newborns with abnormal sulfatide levels and ARSA activity but normal I2S, GALNS and ARSB activities were considered as MLD screen positives. Newborns with abnormal sulfatide levels and ARSA, I2S, GALNS, and ARSB activities were considered as MSD screen positives. Genetic sequencing of the ARSA gene or the SUMF1 gene was performed on a third 3-mm DBS punch accordingly.

While illustrative embodiments have been illustrated and described, it will be appreciated that various changes can be made therein without departing from the spirit and scope of the invention.

1. A method for assaying for arylsulfatase A, comprising:
(a) contacting a sample with a first solution to provide a first solution comprising arylsulfatase A;
(b) isolating arylsulfatase A from the first solution by size exclusion chromatography;
(c) contacting the isolated arylsulfatase A with an arylsulfatase A substrate and incubating the substrate with arylsulfatase A for a time sufficient to provide a solution comprising an arylsulfatase A enzyme product; and
(d) determining the quantity of the arylsulfatase A enzyme product.

2. The method of claim 1, wherein the sample is a dried blood spot.

3. The method of claim 1, wherein the sample is a blood leukocyte sample, a whole blood sample, a plasma sample, a cerebrospinal fluid sample, or a tissue sample.

4. A method for assaying for arylsulfatase A, comprising:
(a) contacting a sample with a solution to provide a solution comprising arylsulfatase A;
(b) contacting the solution comprising arylsulfatase A with an arylsulfatase A substrate, and incubating the substrate with arylsulfatase A for a time sufficient to provide a solution comprising an arylsulfatase A enzyme product; and
(c) determining the quantity of the arylsulfatase A enzyme product.

5. The method of claim 1, wherein the sample is a blood leukocyte sample, a whole blood sample, a plasma sample, a cerebrospinal fluid sample, or a tissue sample.

6-8. (canceled)

9. The method of claim 1, wherein determining the quantities of the arylsulfatase A enzyme product comprises mass spectrometric analysis.

10. The method of claim 1, wherein determining the quantities of the arylsulfatase A enzyme product comprises conducting the product to a mass spectrometer by liquid chromatography or by flow injection.

11. The method of claim 1, wherein the arylsulfatase A substrate is selected from an isotopically-labeled arylsulfatase and a non-isotopically-labeled arylsulfatase A substrate.

12. The method of claim 1, wherein the arylsulfatase A substrate is a deuterated arylsulfatase A substrate.

13. The method of claim 1, wherein the arylsulfatase A substrate is d₃-C18:0-sulfatide.

14. The method of claim 1, wherein the arylsulfatase A enzyme product is d₃-C18:0-galactosyl-ceramide.

15. The method of claim 1, wherein the solution comprising arylsulfatase A substrate further comprises an arylsulfatase A internal standard.

16. The method of claim 1, wherein the arylsulfatase A internal standard is an isotopically-labeled internal standard.

17. The method of claim 1, wherein the arylsulfatase A internal standard is a deuterium-labeled internal standard.

18. The method of claim 1, wherein the aryl sulfatase A internal standard is d₇-C18:0-galactosyl-ceramide.

19. The method of claim 1, wherein the arylsulfatase A substrate is d₃-C18:0-sulfatide and the arylsulfatase A enzyme product is d₃-C18:0-galactosyl-ceramide.

20. The method of claim 1 further comprising using the quantity of the arylsulfatase A enzyme product to determine whether the sample is from a candidate for treatment for a condition associated with arylsulfatase A deficiency.

21. A kit for assaying arylsulfatase A, comprising an arylsulfatase A substrate.

22. The kit of claim 21, wherein the arylsulfatase A substrate is an isotopically-labeled substrate.

23-24. (canceled)

25. The kit of claim 21 further comprising an internal standard for arylsulfatase A.

26-34. (canceled)
